(19) United States
(12) Patent Application Publication
(10) Pub. No.: US 2005/0244902 A1
(43) Pub. Date: Nov. 3, 2005

(54) Methods for determining levels of human b-type natriuretic peptide precursors

(76) Inventors: Jens Peter Gotze, Copenhagen (DK); Jens F. Rehfeld, Charlottenlund (DK)

Correspondence Address: MORRISON & FOERSTER LLP, 3811 VALLEY CENTRE DRIVE, SUITE 500, SAN DIEGO, CA, 92130-2332, US

(21) Appl. No.: 10/510,880

(22) PCT Filed: Apr. 11, 2003
(86) PCT No.: PCT/DK03/00250

§ 371 Date: May 25, 2005

(30) Foreign Application Priority Data
Apr. 11, 2002 (AU) PS 1692

(51) Int. Cl. G01N 33/53; G01N 33/537; G01N 33/543

(52) U.S. Cl. 435/7.92

(57) ABSTRACT

The present invention provides an alternate and more accurate assay for measuring the levels of BNP precursors, or fragments thereof, in a sample by determining the concentration of BNP precursors, or fragments thereof, in a sample obtained from a mammal, the method comprising treating the sample with an agent that cleaves the BNP precursor, and exposing the samplt to an antibody that specifically binds to the cleaved product.

### FIELD OF THE INVENTION

The present invention relates to methods for determining levels of human B-type natriuretic peptide precursors, or fragments thereof, in a sample. In particular, the present invention relates to the use of these methods for determining the risk of a patient of having various conditions, such as cardiac dysfunction, correlated with elevated levels of human B-type natriuretic peptide precursor, or fragments thereof.

### BACKGROUND OF THE INVENTION

Human B-type Natriuretic Peptide (BNP), a member of the cardiac natriuretic peptide family, is a 32 amino acid peptide with potent natriuretic, diuretic and vasodilatatory endocrine functions. The BNP gene is predominantly expressed in the myocytes of the failing heart with BNP increasingly secreted into the circulation in patients with congestive heart failure. Consequently, the diagnostic use of plasma BNP measurements has been studied (Mair et al., 2001). Increased plasma concentrations of BNP are associated with impaired function of the left ventricle disregarding the underlying cause and are therefore valuable in the primary diagnosis of heart failure.

The BNP gene encodes preproBNP, a 134 amino acid residue precursor in which proBNP contains 108 amino acid residues and the bloactive BNP-32 sequence constitutes the C-terminus (FIG. 1). In 1995, Hunt et al. showed that a fragment N-terminal of the active peptide also circulates in plasma and that the concentration increases in heart failure patients. Chromatographic studies have at least suggested the presence of a high molecular weight proBNP peptide (known in the art as proBNP or BNP₁₋₁₀₈) as well as a shorter N-terminal fragment, most likely to be a 1-76 fragment (known in the art as proBNP₁₋₇₆), in plasma from patients with congestive heart failure, however, a complete understanding of the molecular heterogeneity of proBNP-derived peptides in plasma is yet to be realized (Hunt et al., 1995 and 1997a; Schultz et al., 2001).

Several assays directed against both the N-terminal portions of proBNP and bloactive BNP-32 have now been developed (Hunt et al., 1995 and 1997a, b; Schultz et al., 2001; Karl et al., 1999; Hughes et al., 1999; Campbell et al., 2000; U.S. Pat. No. 6,124,430; U.S. Pat. No. 5,786,163) and generally, the plasma concentrations of these portions, like bloactive BNP-32, have been reported to be elevated in patients with heart failure.

However, there remains a troublesome discrepancy between the reported concentrations of various proBNP fragments in healthy subjects as well as in heart failure patients when using the different immunoassays. This hinders to some extent the ability of current BNP-related assays to be used as reliable indicators of cardiac disease states. Accordingly, the present invention provides an alternative assay for accurate quantitation of the levels BNP precursors, or fragments thereof, in a biological sample.

### SUMMARY OF THE INVENTION

The present inventors have developed an alternate assay for measuring the levels of BNP precursors, or fragments thereof, in a sample.

In one aspect, the present invention provides a method for determining the concentration of BNP precursors, or fragments thereof, in a sample obtained from a mammal, the method comprising treating the sample with an agent that cleaves the BNP precursor, and exposing the sample to an antibody that specifically binds to the cleaved product.

The present inventors have found that cleavage of the BNP precursor, or fragments thereof, with an agent that cleaves proteins at basic amino acid residues reduces non-specific immunoreactivity of antibodies which bind the N-terminus of, for example, proBNP₁₋₂₁ and proBNP₁₋₇₆.

Accordingly, in a second aspect the present invention provides a method for determining the concentration of BNP precursors, or C-terminally truncated fragments thereof, in a sample obtained from a mammal, the method comprising treating the sample with an agent that cleaves proteins at basic amino acids, and exposing the sample to an antibody that specifically binds an N-terminus of proBNP₁₋₂₁.

The methods of the present invention have also been used to determine the risk of, or the presence of, a cardiac disease in a patient. Accordingly, in a third aspect the present invention provides a method of predicting or diagnosing a cardiac disease, the method comprising performing the method according to the first aspect, wherein elevated levels of antibody binding are indicative of cardiac dysfunction.

A fourth aspect of the present invention provides a method of predicting or diagnosing a cardiac transplant rejection episode within a patient, the method comprising performing the method according to the first aspect, wherein an increase in antibody binding is an indication of a rejection episode.

A fifth aspect of the present invention provides a method of distinguish between pulmonary and cardiovascular causes of dyspnea, the method comprising performing the method according to the first aspect, wherein elevated levels of antibody binding is an indication of a cardiovascular cause of dyspnea.

A sixth aspect of the present invention provides a method of predicting or diagnosing an ischemic heart disease, the method comprising performing the method according to the first aspect, wherein elevated levels of antibody binding are indicative of ischemic heart disease.

### DETAILED DESCRIPTION OF THE INVENTION

The present inventors have developed an alternate assay for measuring of the levels of BNP precursors, or fragments thereof, in a sample.

In one aspect, the present invention provides a method for determining the concentration of BNP precursors, or fragments thereof, in a sample obtained from a mammal, the method comprising treating the sample with an agent that cleaves the BNP precursor, and exposing the sample to an antibody that specifically binds to the cleaved product.

As the skilled addressee will be aware, in vivo cleavage events may have already produced at least some fragments of the BNP precursor which are identical to those resulting from action of the agent. However, the present invention allows levels of BNP precursor, or fragments thereof, to be analysed regardless of the degree of prohormone processing that has occurred in vivo.

In particular, following cleavage by the agent, a more homogeneous and smaller population of proBNP fragments is achieved. This avoids the potential problem that the same epitope in different fragments is not recognized with the same efficiency when bound to the same antibody. For example, with reference to a preferred embodiment, cleavage with trypsin to produce proBNP₁₋₂₁ circumvents any differential binding of an antibody to this sequence when it forms part of proBNP₁₋₇₆ or proBNP₁₋₁₀₈. In addition, there is the further possible advantage that the cleaved products do not bind potential carrier proteins and/or do not form homo-oligomers.

For the most accurate results, it is preferred that the agent cleaves all potential BNP precursors, or fragments thereof, within the sample.

PreproBNP and Fragments Thereof

FIG. 1 provides the amino acid sequence of preproBNP in human (SEQ ID NO:1), pig (SEQ ID NO:2) and mouse (SEQ ID NO:3). However, at least naturally occurring variants/mutants are also encompassed within the methods of the present invention.

The “pre” region encompasses the N-terminal signal sequence which directs the molecule through the secretory pathway. This sequence is typically removed in the cell through the action of an enzyme generally known as a “signalase”. It is generally accepted in the art that, when considering human preproBNP, the N-terminal 26 amino acids are removed by signalase activity before the polypeptide is packaged in granules and secreted by the cell.

The terms “proBNP” and “BNP precursor” and “proBNP₁₋₁₀₈” are generally used interchangeably herein to refer to the complete BNP prohormone sequence (for human see SEQ ID NO:5). In relation to species other than human, the numbering of “proBNP₁₋₁₀₈” will generally vary. As indicated above, it is generally considered that the first 26 amino acids of human preproBNP are removed by signalase activity. However, the possibility of secreted proBNP molecules N-terminally extended beyond “proBNP₁₋₁₀₈” is not excluded from the present invention.

A number of circulating fragments of preproBNP have been described. These include active BNP (SEQ ID NO:4), proBNP₁₋₁₀₈ (SEQ ID NO:5), and the N-terminal product produced by cleavage of proBNP₁₋₁₀₈, to release the active peptide (SEQ ID NO:4), known in the art as proBNP₁₋₇₆ (SEQ ID NO:6). As used herein the “region of BNP” refers to proBNP₁₋₇₆.

Preferably, the fragment of the BNP precursor is selected from the group consisting of mature BNP or a fragment thereof, and the region of BNP or a fragment thereof. More preferably, the fragment of the region of proBNP is proBNP₁₋₂₁.

The cleavage product can be any fragment of proBNP, which can bind an antibody. In one embodiment, the fragment is at least 6 amino acids in length, such as 7 amino acids in length, 8 amino acids in length or 9 amino acids in length. In another embodiment, the fragment is at least 10 amino acids in length such as 11 amino acids in length, 12 amino acids in length, 13 amino acids in length, 14 amino acids in length, 15 amino acids in length, 16 amino acids in length, 17 amino acids in length, 18 amino acids in length or 19 amino acids in length. In a further embodiment, the fragment is at least 20 amino acids in length, such as 21 amino acids in length, 22 amino acids in length, 23 amino acids in length, 24 amino acids in length, at least 25 amino acids in length, at least 30 amino acids in length, at least 35 amino acids in length, at least 40 amino acids in length, at least 45 amino acids in length, at least 50 amino acids in length, at least 55 amino acids in length or least 60 amino acids in length. Furthermore, it is an object of preferred embodiments to provide fragments, which are less than 76 amino acids in length, such as 75 amino acids in length, 74 amino acids in length, 73 amino acids in length, 72 amino acids in length, 71 amino acids in length, 70 amino acids in length, less than 65 amino acids in length or less than 62 amino acids in length.

Preferably, the antibody binds the N-terminus or the C-terminus of the cleaved product.

Preferably, the antibody binds the N-terminus of proBNP₁₋₂₁.

The sample can be any biological material from the mammal that comprises the BNP precursor or fragments thereof. Preferably, the sample is selected from the group consisting of whole blood, serum, plasma, urine and tissue extracts of the heart and other organs. Most preferably, the sample is plasma extracted from the blood of a mammal.

The agent can be any molecule that cleaves the BNP precursor to produce a fragment which can bind an antibody. Preferably, the agent is an enzyme. More preferably, the enzyme is an endoprotease which cleaves at basic amino acids. More preferably, the endoprotease which cleaves at basic amino acids is a serine protease. More preferably, the serine endoprotease is selected from the group consisting of trypsin, furin, Corin, yeast Kex2, prohormone convertase-1 and prohormone convertase-2. Even more preferably, the serine endoprotease is trypsin.

As the skilled addressee would be aware, the sample could be further purified to remove, for example, lipids and/or nucleic acids and/or proteins which are larger than the BNP precursor. Such sample fractions are also useful for the methods of the present invention. However, it is generally most convenient that the method is performed on the non-fractionated sample.

In a further embodiment, the mammal is selected from the group consisting of humans, horses, apes, pigs, rats, cows, dogs and mice. Preferably, the mammal is a human.

Antibodies

Antibodies useful for the methods of the present invention can be, for example, polyclonal or monoclonal antibodies, chimeric, single chain and humanized antibodies, as well as Fab fragments, or the product of a Fab expression library. Various procedures known in the art may be used for the production of such antibodies and fragments. The antibodies may be made in vivo in suitable laboratory animals or in vitro using recombinant DNA techniques.

As used herein, the term “specifically binds to the cleaved product” refers to the ability of the antibody to recognise the cleaved proBNP fragment whilst not binding to other polypeptides in the sample to a any significant degree to interfere with the capability of the method of the present invention to be used as a diagnostic assay for elevated levels of proBNP, or fragments thereof, in a sample. The antibody may or may not bind to larger precursors of the cleaved fragment (for example proBNP).

Means for preparing and characterizing antibodies are well known in the art (see, for example, Antibodies: A Laboratory Manual, Cold Spring Harbor Laboratory, 1988). The methods for generating polyclonal antibodies are well known in the art. Briefly, polyclonal antisera are prepared by immunizing an animal with the desired antigen and collecting antisera from that immunized animal. A wide range of animal species can be used for the production of antisera. Typically the animal used for production of anti-antisera is a rabbit, a mouse, a rat, a hamster, a guinea pig, a goat, a sheep or a chicken. Because of the relatively large blood volume of rabbits and the ability to produce high-affinity antibodies, a rabbit is a preferred choice for production of polyclonal antibodies.

The amount of antigen composition used in the production of polyclonal antibodies varies upon the nature of the antigen, as well as the animal used for immunization. A variety of routes can be used to administer the antigen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting is repeated until a suitable titre is achieved. When a desired level of immunogenicity is obtained, the immunized animal can be bled and the serum isolated and stored, and/or the animal can be used to generate MAbs (below).

Polyclonal antisera according to present invention are produced against peptides that are predicted to comprise whole, intact epitopes. It is believed that these epitopes are, therefore, more stable in an immunologic sense and thus express a more consistent immunologic target for the immune system. Under this model, the number of potential B-cell clones that will respond to this peptide is considerably smaller and, hence, the homogeneity of the resulting sera will be higher. In various embodiments, the present invention provides for polyclonal antisera where the clonality, i.e., the number of antibody producing clones, that contributes to the production of the antibodies used in an actual RIA setup, is limited to one (mono-clonality) or a few clones (oligo-clonality). Hence, from a functional point of view, the antibodies used in a competitive RIA preferably originate from a single clone (functional MAbs).

MAbs may be readily prepared through use of well-known techniques, such as those exemplified in U.S. Pat. No. 4,196,265. Typically, this technique involves immunizing a suitable animal with a selected antigen composition, e.g., purified or partially purified protein, synthetic protein or fragments thereof. The immunizing composition is administered in a manner effective to stimulate antibody producing cells. Rodents such as mice and rats are preferred animals, however, the use of others animals such as rabbit, sheep or frog cells is possible. The use of rats may provide certain advantages, but mice are preferred, with the BALB/c mouse being most preferred as the most routinely used animal and one that generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer removed. Spleen lymphocytes are obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency and enzyme deficiencies that render then incapable of growing in certain selective media which support the growth of only the desired fused cells, called “hybridomas.”

Selected hybridomas are serially diluted and cloned into individual antibody-producing cell lines, which clones can then be propagated indefinitely to provide MAbs. The cell lines may be exploited for MAb production in two basic ways. A sample of the hybridoma can be injected, usually in the peritoneal cavity, into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such as HPLC or affinity chromatography.

In another embodiment, monoclonal antibodies according to the present invention is a “humanized” monoclonal antibody, produced by techniques well-known in the art. That is, mouse complementary determining regions (“CDRs”) are transferred from heavy and light V-chains of the mouse Ig into a human V-domain, followed by the replacement of some human residues in the framework regions of their murine counterparts.

As stated above, the monoclonal antibodies and fragments thereof useful for the methods of the present invention can be multiplied according to in vitro and in vivo methods well-known in the art. Multiplication in vitro is carried out in suitable culture media such as Dulbecco's modified Eagle medium or RPMI 1640 medium, optionally replenished by a mammalian serum such as fetal calf serum or trace elements and growth-sustaining supplements, e.g., feeder cells, such as normal mouse peritoneal exudate cells, spleen cells, bone marrow macrophages or the like. In vitro production provides relatively pure antibody preparations and allows scale-up to give large amounts of the desired antibodies. Techniques for large scale hybridoma cultivation under tissue culture conditions are known in the art and include homogenous suspension culture, e.g., in an airlift reactor or in a continuous stirrer reactor or mmobilized or entrapped cell culture.

Fragments of a monoclonal antibody can be obtained from monoclonal antibodies produced as described above, by methods which include digestion with enzymes such as pepsin or papain and/or cleavage of disulfide bonds by chemical reduction. Alternatively, monoclonal antibody fragments encompassed by the present invention can be synthesized using an automated peptide synthesizer, or they may be produced manually using techniques well known in the art.

Antibody binding to cleaved BNP precursors can be detected by any means known in the art. Preferably, antibody binding is detected by an assay selected from the group consisting of radioimmunoassay (RIA), enzyme-linked immunosorbent assay (ELISA), fluoroimmunoassay, immunofluorometric assay, immunoradiometric assay and mass spectrometry, such as a MALDI-TOF spectrometry. Most preferably, antibody binding to cleavage products is detected by RIA or ELISA.

Immunoassays

Immunoassays, in their most simple and direct sense, are binding assays. Antibody binding to a cleavage product can be detected by any immunoassay means known in the art. Preferably, antibody binding is detected by an assay selected from the group consisting of protein microarray assay, radioimmunoassay (RIA), enzyme-linked immunosorbent assay (ELISA), fluoroimmunoassay, immunofluorometric assay, and immunoradiometric assay.

Immunoradiometric assays as they can be applied to antibodies directed against proBNP are generally described in Kono et al. (1993) and Clerico et al. (1998). Such assays can be used in the methods of the present invention.

Most preferably, antibody binding is detected by RIA or ELISA.

Radioimmunoassay (RIA)

RIA is a highly sensitive technique that can detect antigen or antibody at concentrations less than 0.001 μg/ml.

The principle of RIA involves competitive binding of radiolabelled antigen and unlabelled antigen to a high-affinity antibody. The antigen is generally labelled with a gamma-emitting isotope such as ¹²⁵I. The labelled antigen is mixed with antibody at a concentration that just saturates the antigen-binding sites of the antibody molecule, and then increasing amounts of unlabelled antigen of unknown concentration are added. The antibody does not distinguish labelled from unlabelled antigen, and so the two kinds of antigen compete for available binding sites on the antibody. With Increasing concentrations of unlabelled antigen, more labelled antigen will be displaced from the binding sites. By measuring the amount of labelled antigen free in solution, it is possible to determine the concentration of unlabeled antigen.

Several methods have been developed for separating the bound antigen from the free antigen in RIA. One method involves precipitating the Ag-Ab complex with a secondary antl-isotype antiserum. For example, if the Ag-Ab complex contains rabbit IgG antibody, then goat anti-rabbit IgG can precipitate the complex. Another method makes use of the fact that protein A of Staphylococcus aureus has high affinity for IgG. If the complex contains an IgG antibody, the complex can be precipitated by mixing with formalin-killed S. aureus. After removal of the complex by either of these methods, the amount of free labelled antigen remaining in the supernatant can be quantitated in a gamma counter. A standard curve is then plotted of the percentage of bound labelled antigen versus known concentrations of unlabeled antigen. Once a standard curve had been plotted, unknown concentrations of the unlabeled antigen can be determined from the standard curve.

Various solid-phase RIAs have been developed that make it easier to separate the Ag-Ab complex from the unbound antigen. In some cases the antibody is covalently cross-linked to Sepharose® beads. The amount of radiolabelled antigen bound to the beads can be quantitated after the beads have been centrifuged and washed. Alternatively, the antibody can be immobilized on polystyrene or polyvinylchloride and the amount of free labelled antigen in the supernatant can be determined in a gamma counter. In another approach, the antibody is immobilized on the walls of microtiter wells. This procedure is well suited for determining the concentration of a particular antigen in large numbers of samples.

Enzyme-Linked Immunosorbent Assay (ELISA)

EUSA relies on a similar principle to RIA but depends on an enzyme rather than a radioactive label. More specifically, an enzyme conjugated to the antibody is able to generate a detectable signal in the presence of a suitable substrate.

In one exemplary ELISA, the antibodies useful for the methods of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody which binds the antigen that is linked to a detectable label. This type of ELISA is a simple “sandwich ELISA”. Detection may also be achieved by the addition of a second antibody that binds the antigen, followed by the addition of a third antibody that has binding affinity for the second antibody, with the third antibody being linked to a detectable label.

In another exemplary ELISA, the samples suspected of containing the antigen are immobilized onto the well surface and then contacted with the antibodies. After binding and appropriate washing, the bound immune complexes are detected. Where the initial antibodies are linked to a detectable label, the immune complexes may be detected directly. Again, the immune complexes may be detected using a second antibody that has binding affinity for the first antibody, with the second antibody being linked to a detectable label.

Competition ELISAs are also possible in which test samples compete for binding with known amounts of labelled antigens or antibodies. The amount of reactive species in the unknown sample is determined by mixing the sample with the known labelled species before or during incubation with coated wells. The presence of reactive species in the sample acts to reduce the amount of labelled species available for binding to the well and thus reduces the ultimate signal.

Irrespective of the format employed, ELISAs have certain features in common, such as coating, incubating or binding, washing to remove non-specifically bound species, and detecting the bound immune complexes. These are described as below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of the antigen or the antibody to the well, coating with a non-reactive material to reduce background, and washing to remove unbound material, the immobilizing surface is contacted with the clinical or biological sample to be tested under conditions effective to allow immune complex (antigen/antibody) formation. Detection of the immune complex then requires a labelled secondary binding ligand or antibody, or a secondary binding ligand or antibody in conjunction with a labelled tertiary antibody or third binding ligand.

“Under conditions effective to allow immune complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The suitable conditions also mean that the incubation is at a temperature and for a period of time sufficient to allow effective binding. Incubation steps are typically from about 1 to 2 to 4 hours, at temperatures preferably in the order of 25 to 27° C., or may be overnight at about 4° C. or so.

Following all incubation steps in an ELISA, the contacted surface is washed so as to remove non-complexed material. Washing often includes washing with a solution of PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immune complex with a urease, glucose oxidase, alkaline phosphatase or hydrogen peroxidase-conjugated antibody for a period of time and under conditions that favor the development of further immune complex formation, e.g., incubation for 2 hours at room temperature in a PBS-containing solution such as PBS-Tween.

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

Alternatively, the label may be a chemilluminescent one. The use of such labels is described in U.S. Pat. Nos. 5,310,687, 5,238,808 and 5,221,605.

Protein Microarray Assay Another embodiment of the present invention relates to detecting the amount of at least one form of proBNP present in the sample detected specifically using a protein microarray assay. This means that the capturing of the at least one form of proBNP is performed using antibodies or fragments thereof and wherein the detection is performed using a mass spectrometer, such as a MALDI-TOF spectrometer. Furthermore, the binding of at least one form of proBNP onto a surface can be utilised as catching mechanism and the detection can be made using a mass spectrometer, such as a SELDI-TOF spectrometer.

The present inventors have found that cleavage of the BNP precursor, or fragments thereof, with an agent that cleaves proteins at basic amino acid residues reduces non-specific immunoreactivity of antibodies which bind the N-terminus of, for example, proBNP₁₋₂₁ proBNP₁₋₄₆, proBNP₁₋₅₄, proBNP₁₋₆₂, proBNP₁₋₆₅, proBNP₁₋₇₃ and proBNP₁₋₇₆.

Accordingly, in a second aspect the present invention provides a method for determining the concentration of BNP precursors, or C-terminally truncated fragments thereof, in a sample obtained from a mammal, the method comprising treating the sample with an agent that cleaves proteins at basic amino acids, and exposing the sample to an antibody that specifically binds an N-terminus of a BNP precursor such as proBNP₁₋₂₁.

With regard to the second aspect, the agent can be any molecule that cleaves proteins at basic amino acids. Preferably, the agent is an enzyme. More preferably, the enzyme is a serine protease. Even more preferably, the serine protease is trypsin.

Cardiac Dysfunction

Concentrations of the BNP precursor, and various fragments thereof, have been shown to be correlated with cardiac dysfunction. Thus, the methods of the present invention can be used to determine the risk of, or the presence of, a cardiac related disease in a patient.

Accordingly, in a third aspect the present invention provides a method of predicting or diagnosing a cardiac disease, the method comprising performing the method according to the first aspect, wherein elevated levels of antibody binding are indicative of cardiac dysfunction.

As the skilled addressee will be aware any medical evaluation based on a prognostic and/or diagnostic marker such as the concentration of a BNP precursor in a sample would require a correlation to a reference value. Some disease could have its own preferred reference value and some diseases could have a shared value which would be indicative for these diseases in general.

The reference value can be calculated from a pool of samples from individuals with the disease and by comparison with a pool of samples from healthy individuals, a range for positive and negative calls can be made. Another possibility is to set a reference value based on a pool of samples from various phases or stages of the disease to determine the progression or a stage of the disease. It may even be desirable to set reference values for prognosis of the disease. The reference value can be calculated as a mean or a median value of each concentration obtained.

As indicated in the examples below an elevated level of antibody binding level of proBNP above 15 pmol/L is normally a strong indication of a cardiac disease. In most individuals a level between 0-15 pmol/L proBNP is considered normal. This may however vary so levels below 15 pmol/L, such as 14 pmol/L, 13 pmol/L, 12 pmol/L, 11 pmol/L and pmol/L can in some case still be indicative of a disease state. Especially in elderly people, the individual level of proBNP varies and should preferably be correlated to a reference value. However, it is an object of preferred embodiments of the present invention to diagnose a cardiac disease based on an elevated level of antibody binding above 15 pmol/L. Levels above 15 pmol/L should be followed by further cardiac examination, such as but not limited to coronary anglography procedure. If a level below 15 pmol/L is observed the individual could be put under further observation or correlated to more specific reference values.

In a preferred embodiment of the third aspect, the cardiac dysfunction is selected from the group consisting of: congestive heart failure, impaired function of the left ventricle, cardiac failure after myocardial infarction, arrhythmogennic right dysplasia, congenital heart disease, obstructive hypertrophic cardiomyopathy, and cardiac related acute dyspnea.

Cardiac Allograft Rejection

It has also previously been established that concentrations of the BNP precursor, and various fragments thereof, can be an indicator for the prediction and diagnosis of cardiac allograft rejection in a patient who has been subjected to such an allograft (U.S. Pat. No. 6,117,644). The methods of the present invention can be used for this purpose.

Accordingly, in a fourth aspect the present invention provides a method of predicting or diagnosing a cardiac transplant rejection episode within a patient, the method comprising performing the method according to the first aspect, wherein an increase in antibody binding is an indication of a rejection episode.

Distriminating between Pulmonary and Cardiovascular Causes Of Dyspnea

The present inventors have also shown that the methods of the present invention can be used as a simple diagnostic test to distinguish between pulmonary and cardiovascular causes of dyspnea.

By use of the present invention the present inventors measured the plasma concentrations of proBNP in terminal parenchymal lung disease patients with normal left ventricular function referred for evaluation for lung transplantation undergoing right heart catheterization (n=50) and the results related to the hemodynamic variables obtained from the catheterization.

Plasma proBNP concentrations were unaffected in patients with terminal parenchymal lung disease and normal left ventricular function (median 2.5 pmol/l, range 0.0-22.0, upper reference limit: 15.0 pmol/l). In contrast, patients with primary pulmonary hypertension displayed more than 40-fold elevation in plasma proBNP concentrations (median 107 pmol/l vs. median 2.5 pmol/l, P<0.0001). Regional vascular proBNP concentrations revealed the heart as the secretory site.

These findings strongly support the contention of natriuretic peptide measurements as efficient markers for cardiovascular causes of dyspnea. Moreover, our results eliminate natriuretic peptides as markers of moderate pulmonary hypertension in patients with terminal parenchymal lung disease.

Thus, in a fifth aspect the present invention provides a method of distinguish between pulmonary and cardiovascular causes of dyspnea, the method comprising performing the method according to the first aspect, wherein elevated levels of antibody binding is an indication of cardiovascular causes of dyspnea.

Cardiac BNP Expression is Increased in Myocardial Ischemia

A sixth aspect of the present invention provides a method of predicting or diagnosing a ischemic heart disease, the method comprising performing the method according to the first aspect, wherein elevated levels of antibody binding are indicative of a ischemic heart disease.

Congestive heart failure is accompanied by increased cardiac BNP gene expression and augmented plasma concentrations of BNP and its precursor, proBNP. Recent studies have shown that plasma concentrations of BNP and proBNP are also elevated in patients presenting with unstable coronary syndrome or myocardial infarction. The present inventors have investigated whether myocardial ischemia, in the absence of overt heart failure, may be another mechanism for increased cardiac BNP gene expression and elevated plasma BNP and proBNP concentrations, data presented in example 3.

It is known that cardiac BNP gene expression is increased in failing hearts and that plasma measurements of BNP and proBNP are markers of impaired function of the left ventricle. Recently, it was shown that plasma BNP and proBNP concentrations provides prognostic information of later development of heart failure during an acute myocardial infarction and furthermore are prognostic markers of morbidity and death in patients presenting with acute coronary syndrome. Until now, it has however been not been explained precisely how an elevated plasma concentration of BNP or proBNP is a marker of manifest heart failure and in other studies precedes and even predicts subsequent cardiac dysfunction. By use of the present invention the present inventors have shown that myocardial ischemia per se causes the increase in plasma BNP and proBNP concentrations. This mechanism most likely reflects an increased cardiac BNP gene expression in the ischemic left ventricle, since plasma BNP and proBNP concentrations were closely associated with ventricular BNP mRNA expression. In contrast, the present inventors found no association between plasma BNP and proBNP concentrations with atrial BNP mRNA expression, despite a markedly higher BNP mRNA content in the atrial than the ventricular biopsies. This most likely reflects a different processing and storage of proBNP peptides in atrial and ventricular myocytes as well as the smaller atrial mass compared to the large ventricular myocardium. In conclusion, the present inventors have shown that ventricular BNP gene expression is increased in myocardial ischemia resulting in increased plasma concentrations of BNP and proBNP. Hence, elevated BNP and proBNP concentrations are not only markers of heart failure but also of cardiac ischemia.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention will hereinafter be described by way of the following non-limiting Figures and Examples.

### BRIEF DESCRIPTION OF THE ACCOMPANYING DRAWINGS

FIG. 1. Amino acid sequences of the preproBNP precursor in mouse (SEQ ID NO: 3), pig (SEQ ID NO: 2) and human (SEQ ID NO:3) (panel A). Bold amino acids mark homology with the human sequence. The N-terminal prosequence 1-10 and the known human bloactive peptide (residues 103-134) are underlined. Panel B illustrates proBNP and peptide fragments with the dark rings representing basic arginyl residues. Fragment 1-76 and the fragment 1-21 are outlined, as is the bioactive BNP-32.

FIG. 2. Chromatographic profile of plasma from healthy subjects. Panel A shows the immunoreactivity of eluted non-treated plasma whereas panel B shows the immunoreactivity in the collected fractions after tryptic treatment.

FIG. 3. Chromatographic profile of heart failure plasma. Panel A shows plasma applied directly to the column and panel B shows the plasma profile when initially extracted using Sep-pak C1 S cartridges. Open circles mark untreated fractions and dark circles the trypsin-treated fractions.

FIG. 4. Heart failure plasma treated with trypsin and subjected to G-50 Superfine gel filtration chromatography (dark circles). The elution profile of synthetic proBNP 1-21 is also shown (open circles).

FIG. 5. N-terminal proBNP in healthy subjects. Subjects were divided into the following groups: I: Age 23-50 (n=65), II: Age 51-65 (n=25), III: Elderly with no symptoms of cardiopulmonary disease, age 66-88 (n=18), IV: Elderly with no objective findings of cardiopulmonary disease age, 60-79 (n=18).

FIG. 6. N-terminal proBNP in heart failure patients (dark circles, n=16) and in an age-matched control group (open circles, n=90). One heart failure patient had a plasma proBNP concentration of 659 pmol/L and is not plotted on this figure. The broken line indicates the calculated upper reference limit (15 pmol/L).

FIG. 7. Hemodynamic characteristics of patients with terminal parenchymal lung disease (n=44) and primary pulmonary hypertension (n=6). Panel A, mean pulmonary artery pressure (mPAP); Panel B, cardiac index; Panel C, right ventricular ejection fraction (RVEF); Panel D, pulmonary vascular resistance (PVR).

FIG. 8. Plasma concentrations of proBNP in the subgroups of terminal parenchymal lung disease patients (n=44) and patients with primary pulmonary hypertension (n=6). The dashed lines represent upper reference limits for plasma concentrations (horizontal line) and mean pulmonary artery pressure (vertical line).

FIG. 9. Panel A shows linear regression of plasma concentrations of proBNP (Log) to mean right atrial pressure (mRAP, r²=0.21, P<0.005) or mean pulmonary artery pressure (mPAP) in panel B (r²=0.50, P<0.0001). The dashed lines represent upper reference limits for plasma concentrations (horizontal lines) and mean right atrial or pulmonary artery pressure (vertical lines).

FIG. 10. Patients (n=50) were divided into 3 groups depending on mean pulmonary artery pressure (0-20, 21-60, <61 mmHg). Data are expressed as medians (line) and the dashed lines mark the upper reference limits of propeptide in plasma. ** represents P<0.01, *** P<0.001.

FIG. 11. Linear regression of plasma proBNP and pulmonary artery oxygen saturation (SᵥO₂, r²=0.45, P<0.005) and cardiac index (r²=0.47, P<0.005) in 14 patients with terminal parenchymal disease and 5 PPH patients.

FIG. 12. Plasma proBNP In the femoral vein, pulmonary and femoral artery in patients with elevated plasma proBNP concentrations (n=10). *represents a P value<0.05, n.s.=not significant.

FIG. 13. Left ventricular ejection fraction and plasma BNP and proBNP concentrations.

Panel A: Left ventricular ejection fraction as determined by ventriculography in coronary artery disease patients undergoing percutaneous intervention (PCI), coronary artery bypass grafting surgery (CABG), heart failure patients (CHF) without coronary artery disease, and normal individuals without coronary artery disease or left ventricular dysfunction (controls).

Panel B: Plasma BNP (closed circles) and proBNP (open circles) concentrations in the 4 groups. Horizontal lines indicate median values and each point represents the values of an individual patient.

FIG. 14. Cardiac ventricular BNP mRNA content, and the atrial mRNA content in patients with coronary artery disease.

Panel A: The association between plasma BNP concentration and left ventricular BNP mRNA content in CABG patients.

Panel B: Lack of association between plasma BNP concentration and right atrial BNP mRNA content. Each point represents the values of an individual patient. The r-values were derived from linear regression analysis of logarithmically transformed data.

FIG. 15. Schematic diagram of the proposed mechanism involved in regulation of ventricular BNP gene expression and increased plasma concentrations of BNP and proBNP.

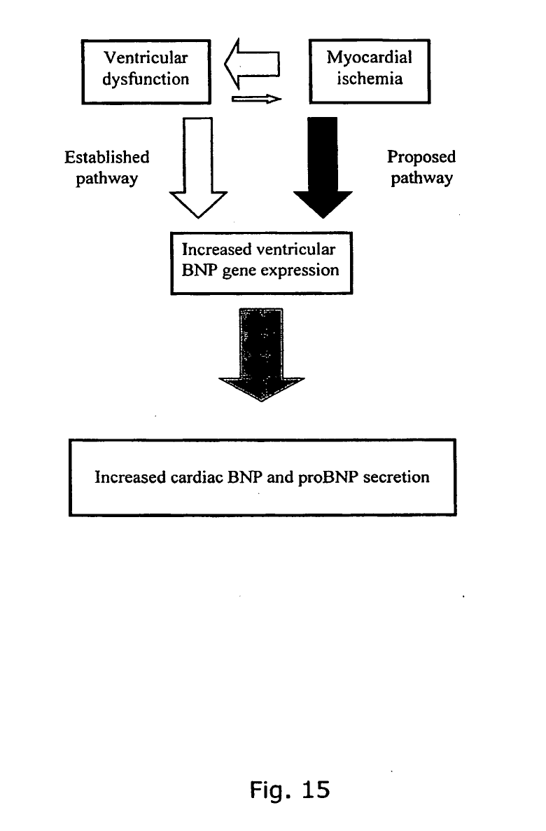

### KEY TO THE SEQUENCE LISTING

SEQ ID NO: 1—Human preproBNP.

SEQ ID NO: 2—Porcine preproBNP.

SEQ ID NO: 3—Murine preproBNP.

SEQ ID NO: 4—Human BNP (active peptide).

SEQ ID NO: 5—Human proBNP₁₋₁₀₈.

SEQ ID NO: 6—Human proBNP₁₋₇₆.

SEQ ID NO: 7—Human proBNP₁₋₂₁.

### EXAMPLES

### Example 1

### Development and Characterization of an Assay for proBNP in Human Plasma

Materials & Methods

Peptides

Human proBNP 1-21, the corresponding N-terminal 1-10 sequence extended C-terminally with a tyrosyl residue for tracer preparation, and the same peptide extended C-terminally with cysteine for directional carrier coupling were custom synthesized (Cambridge Biochemical Research Ltd, Cheshire, UK). An N-terminal truncated fragment of proBNP 1-21 was obtained by controlled cleavage in an automated protein sequencer (Johnsen and Kastrup, 1994). The purity and content of the peptides were verified by reversed-phase HPLC, amino acid analysis and mass spectrometry.

Antiserum

An antibody directed against sequence 1-10 of human proBNP was produced using 10 mg of the 1-10 fragment extended C-terminally with cysteine and coupled to 20 mg bovine serum albumin using the m-maleimidobenzoyi-N-hydroxysuccinimide ester conjugation method. The coupled product was dissolved in 20 ml of distilled water (conjugate solution). The antigen solution (2 ml) was mixed with 3 ml of isotonic saline and emulsified with an equal volume of complete Freund's adjuvant (The State Serum Institute, Copenhagen, Denmark) and used for the first immunization. For booster injections, 1 ml of the antigen solution was mixed with 4 ml of saline and an equal volume of incomplete Freund's adjuvant. Eight random-bred white Danish rabbits were immunized subcutaneously over the lower back at 8-week intervals. Twenty ml of blood was collected from an ear vein 14 days after each immunization, and the serum was stored at −20° C.

Preparation of Tracer

The tyrosine-extended 1-10 fragment (4.5 nmol) was iodinated using a mild chloramine-T method as previously described (Stadil et al., 1972) and subsequently purified on reversed-phase HPLC (Pierce C₈ column, RP-300, 4.6×220 mm) and eluted by a linear ethanol gradient (5-30%) in 1% trifluoroacetic acid. The gradient was selected to ensure separation of the non-labeled peptide from the iodinated tracer. Fractions (1 mL) were collected at a flow rate of 1.0 mL/min. To evaluate the chromatographic separation of labelled and non-labelled peptides, 1 mL of the monoiodinated peak fraction was mixed with 10 pmol of nonlabeled proBNP 1-10Tyr and reapplied to the HPLC-column. The radioactivity and immunoreactivity was thereafter measured. The specific tracer radioactivity was determined by self-displacement (Morris et al., 1976). Peptide standards were made from synthetic proBNP 1-10Tyr in a Tris buffer containing 0.2% human serum albumin (pH 8.5).

Enzymatic Treatment of Plasma

Plasma for measurement was initially treated with trypsin TPCK (Worthington Biochemical Cooperation, Lakewood, N,1, USA). Briefly, plasma samples and standards (200 μL) were mixed with 0.1 mol/L sodium phosphate buffer (pH 7.5) containing trypsin to a final concentration of 2 g/L of trypsin in the incubation mixture. The mixture was incubated at room temperature for 30 min and immediately after boiled for 10 min to terminate the enzymatic reaction. Trypsin-treated samples and standards were then stored at −80° C. Samples were centrifuged for 10 min at 3000 g before assay setup with only the supernatant used in the following RIA.

Plasma Samples

For establishment of a reference interval, 90 healthy volunteers with no medical history or symptoms of systemic or cardiac disease were enrolled. They were divided into two groups: Age 23-50 (65 subjects, median age 33, 28 females, 37 males) and age 51-65 (25 subjects, median age 53, 14 females, 11 males). Blood samples were drawn after a 20 min rest while seated from fasting subjects. A group consisting of 18 elderly subjects (median age 72 (66-88), 10 females, 8 males) with no history of cardiac disease or symptoms were recruited from a local dancing club. Another group of 18 elderly (median age 68 (60-79), 9 females, 9 males) with no history of cardiac disease nor symptoms and signs were accessed using echocardiography, blood pressure measurement, exercise testing (ergometry), pulmonary function test and blood screening. They were all without findings of cardiopulmonary disease. Finally, 16 stable heart failure patients referred for evaluation with respect to cardiac transplantation (median age 55 (30-68), 6 females, 10 males) with no biochemical signs of renal disease were examined. The heart failure patients were classified in NYHA group II-IV and all had a reduced left ventricular ejection fraction (median 30%, range 15-40). Blood samples were drawn from a cubital vein and plasma was stored at −80° C. until analysis. The local medical ethical committee approved the use of human plasma, and Informed consent was obtained from all subjects and patients (KF01-231/99).

Radioimmunoassay (RIA) Procedure

For RIA, 150 μL of trypsin-treated standards and plasma samples were mixed with a Tris buffer (100 μL) containing 0.2% human serum albumin, tracer peptide (1000 counts/min) and antiserum (final dilution 1:150000). Synthetic proBNP 1-10Tyr was used as standards. After 5 days of incubation at 4° C., the antibody-bound and free tracer was separated by adding 2 mL of plasma-coated charcoal (15 g charcoal/L in a 10% plasma Tris buffer), left for 10 min at room temperature and centrifuged. Controls, buffer blanks and sample blanks (without antiserum added) were included in the assays and samples were always assayed in duplicate. The reliability of the assay was evaluated with respect to sensitivity (detection limit), specificity, precision (intra- and interassay reproducibility) and accuracy.

Chromatography

Plasma was applied to a 2000×10 mm Sephadex G-50 Superfine column (Pharmacia, Uppsala, Sweden) and eluted at 4° C. with a Tris buffer containing 0.2% human serum albumin at pH 8.5 (flow rate 4 ml/h). The columns were calibrated with synthetic proBNP 1-21 and void and total volumes were determined by eluting ¹²⁵I-albumin and ²²NaCL, respectively.

Statistical Analysis

Results are expressed as medians and ranges. Calculation of a reference interval was performed by the non-parametrical method according to Reed et al (Reed et al., 1971). Statistical comparison was performed by the Mann-Whitney non-parametrical test and two-tailed P values less than 0.05 were considered significant.

Results

Radioiodination

The incorporation of ¹²⁵I in the tyrosine-extended proBNP 1-10 varied from 80 to 95%. Labelled and nonlabelled peptide were completely separated and the dilution curves for labelled and unlabelled antigen were parallel. The specific activity of the tracer was estimated by self-displacement to be 3.90 Cl/μmol.

Antiserum Evaluation

All rabbits responded to the immunization, and antiserum 98192 was chosen for further characterization due to high titer (1:150000) and avidity. Hence, binding affinity of this antiserum, expressed by the effective equilibrium constant (K^{o}_{aff}) was 0.45×10¹² L/mol, corresponding to a detection limit of 0.07 pmol/L. The detection limit, when calculated as the mean of 10 replicates of 0-standard-3 SD, was 0.20 pmol/L. The index of heterogeneity was calculated to 1.10 according to Sips (1948), indicating that the ligand is highly homogenous and that the antiserum acts as a solution of monoclonal antibodies (Rehfeld, 1988).

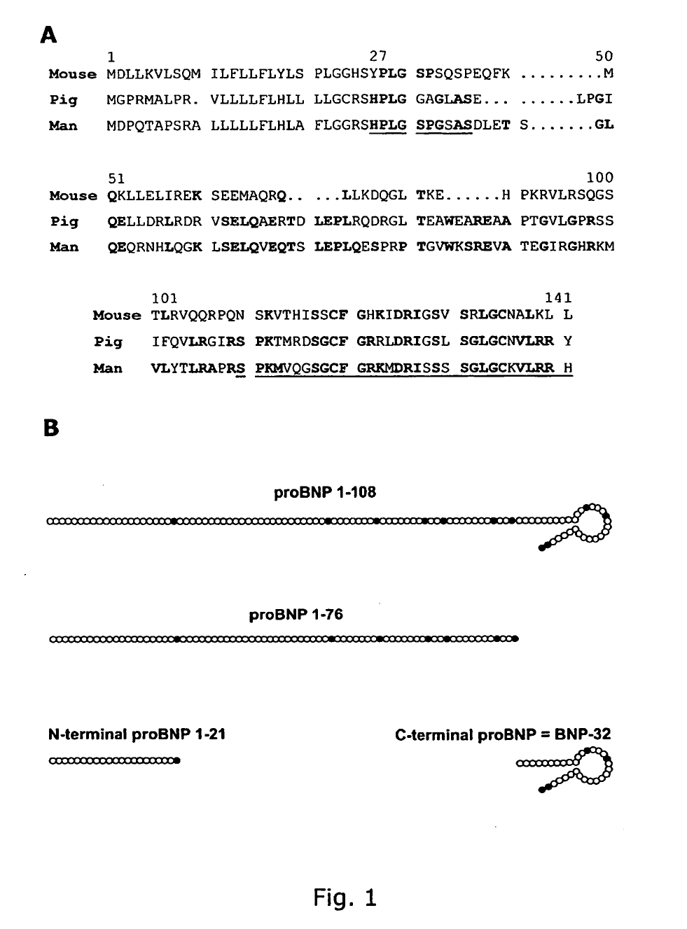

The specificity of the antisera was expressed as the ratio of median inhibitory dose (ID₅₀) for the truncated N-terminal proBNP peptide in tracer displacement. Removal of the N-terminal histidine decreased the binding grossly (0.001) and removal of two N-terminal amino acids, histidine and proline, decreased the binding further (0.0006). Consequently, the two N-terminal amino acids constitute an essential part of the epitope for antiserum 98192. The specificity was further evaluated by measuring N-terminal proBNP concentrations in heart extracts from mouse and pig (FIG. 1). No immunoreactivity could be detected in either species, indicating that also amino acids in position 5-6, serine and proline, in the human sequence are necessary for antibody binding. The computer program FASTA was used to search the SwissProt database for amino acid sequences resembling human N-terminal proBNP. No relevant sequences other than the corresponding N-terminal proBNP sequences of other mammals were found.

Assay Reliability

Dilution curves for trypsin-treated plasma samples were parallel with the calibrator curve (data not shown), indicating that the antiserum affinity to native peptide antigen was equal to that of the synthetic peptides. The inter-assay coefficients of variation of replicate samples were 20% at 16 pmol/L, 8% at 70 pmol/L and 10% at 145 pmol/L (n=10). The inter-assay variation was 12% at 13 pmol/L, 7% at 75 pmol/L and 5% at 130 pmol/L (n=10). The dilution of plasma samples with high concentrations of endogenous N-terminal proBNP as well as mixing plasma samples with high and low concentrations of N-terminal proBNP (as determined by the present assay) showed a high degree of correlation with deviations <15% from the calculated concentrations. The measuring range of the assay was 0-250 pmol/L.

Antiserum Specificity Evaluated by Chromatography of Plasma

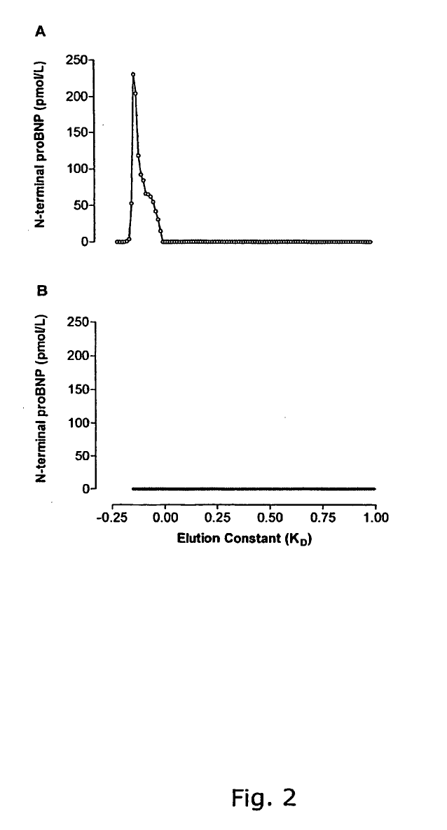

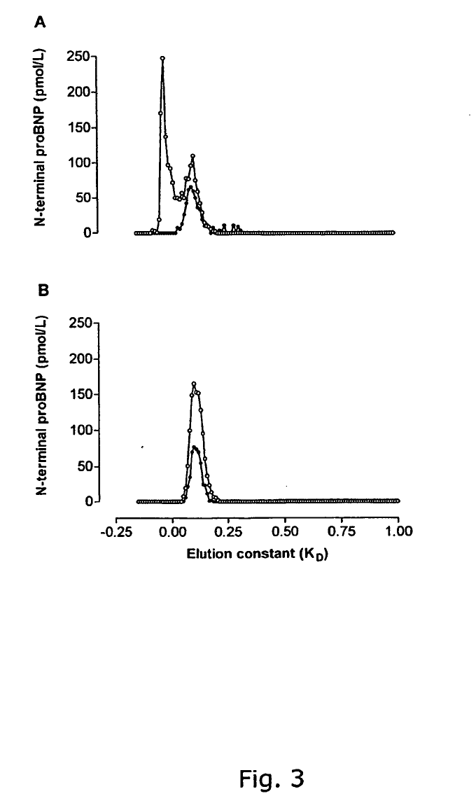

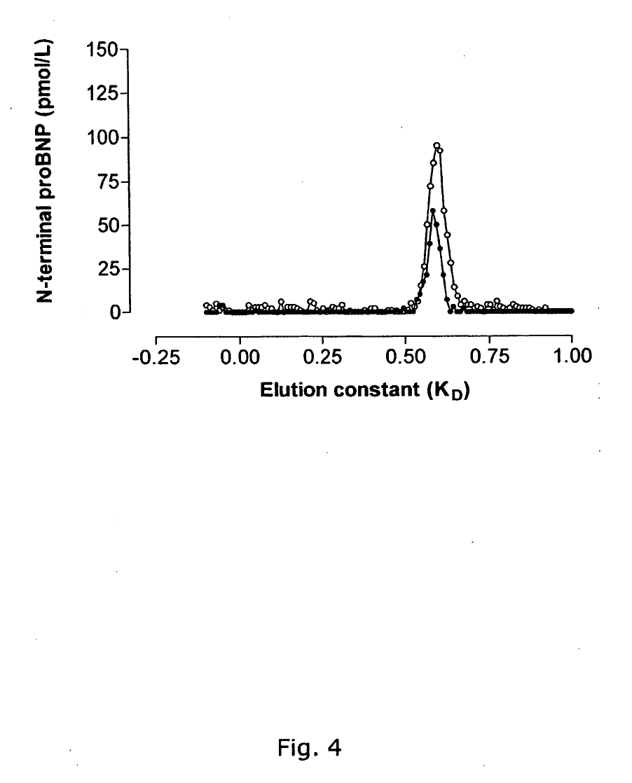

Gel chromatography of normal plasma revealed apparent immunoreactivity eluting in the void volume position. The same fractions proved devoid of immunoreactivity after treatment with trypsin (FIG. 2). Chromatography of plasma from heart failure patients displayed two peaks of immunoreactivity, but after tryptic cleavage of the fractions, only one peak was preserved (K_{D}=0.10, FIG. 3A). When plasma from heart failure patients were extracted using Sep-pak C18 cartridges (Millipore Waters, Milford, Mass., USA) prior to gel chromatography, the immunoreactivity eluted as a single peak in the same position as the peak after trypsin treatment of the fractions (FIG. 3B), but the total immunoreactivity in the peak after trypsin-treatment was reduced. Finally, immunoreactivity of trypsin-treated heart failure plasma subjected to chromatography eluted as a single peak corresponding to the position of the synthetic proBNP 1-21 calibrator peptide (K_{D}=0.59, FIG. 4).

N-Terminal proBNP Stability in Plasma

Heart failure plasma left at room temperature for 24 hours and then treated with trypsin did not reveal any significant decrease in endogenous N-terminal proBNP concentrations (127 to 105 pmol/L, n=5). Likewise, initial trypsin treatment of plasma followed by consequent incubation for 24 hours at room temperature did not lead to degradation of endogenous peptide (128 to 125 pmol/L, n=5).

N-Terminal proBNP in Healthy Subjects

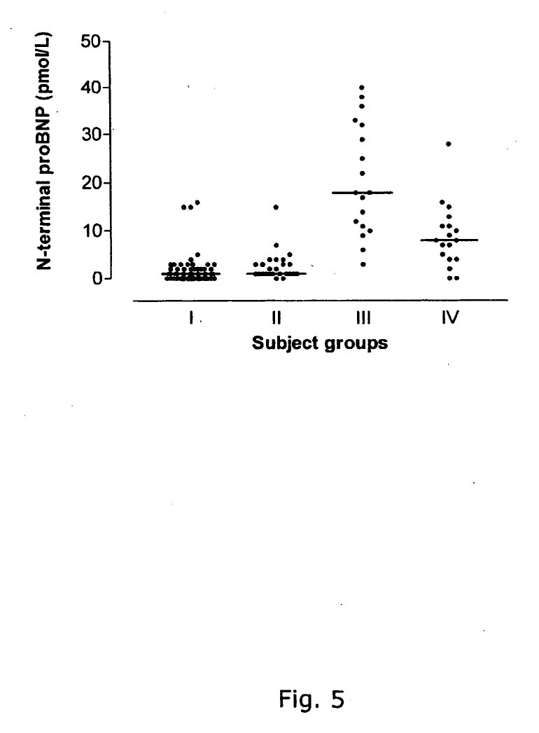

The concentration of N-terminal proBNP in plasma after trypsin treatment is shown in FIG. 5. The medians differed significantly between the 23-50 and 51-65 years age groups (1.0 pmol/L (0-16, n=65) versus 2.0 pmol/L (0-15, n=25), p<0.01), as did the medians between the 51-65 years age group and the 66-88 years age group (22 pmol/L (3-40, n=18), p<0.0001). The plasma concentration in the selected group of elderly without objective signs of cardiopulmonary disease, age 60-79 years, was lower when compared to the age-matched group of healthy elderly only asked for symptoms (8 pmol/L (4-28, n=18) versus 22 pmol/L (3-40, n=18), P<0.0005). There was no significant concentration difference between males and females regardless of age. A histogram of the obtained concentrations revealed that the N-terminal proBNP measurements were not from a single distribution. Therefore, the reference interval was determined from subjects between 23-65 of age, where the distribution was homogenous. Given the non-Gaussian distribution and that it was not possible to transform data to fit a Gaussian distribution, the reference interval was determined by a non-parametrical method using rank numbers 25 and the upper reference limit (97.5 percentile) could then be calculated to 15 pmol/L (confidence interval 9-16 pmol/L, n=90) for subjects 23-65 years of age.

N-Terminal proBNP in Heart Failure Plasma

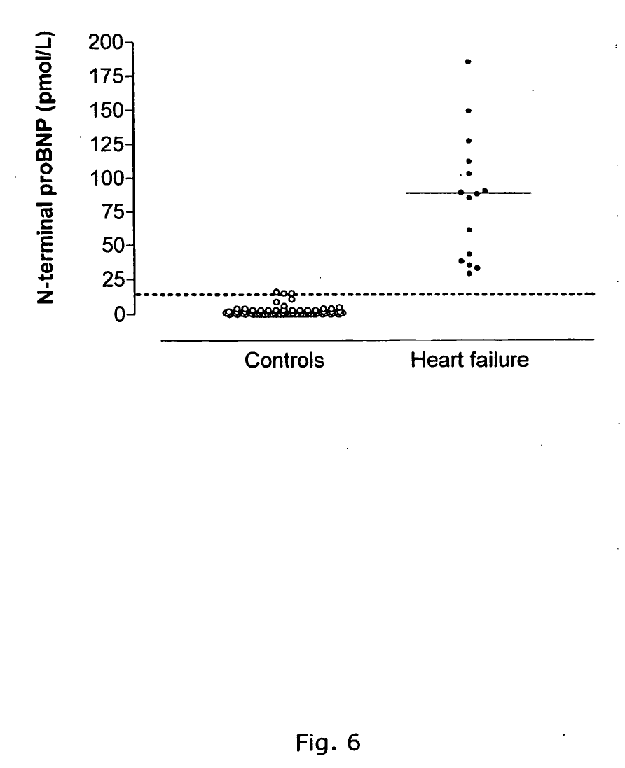

The plasma concentrations in heart failure patients are shown in FIG. 6. N-terminal proBNP concentrations were significantly elevated when compared to the age-matched group of healthy subjects (89 pmol/L (29-659, n=16) versus 1.0 pmol/L (0-16, n=90), p<0.0001) and always higher than the upper reference limit (FIG. 6).

Discussion

The present study describes the development and characterization of an assay for proBNP In human plasma. The antiserum produced was of high avidity and homogeneity; and when used in combination with a monoiodinated tracer, a sensitive and specific assay was obtained. Furthermore, we have applied cleavage of proBNP, thereby cleaving endogenous proBNP and its N-terminal fragments in plasma to the small 1-21 fragment. This step allows accurate quantitation of proBNP and its products irrespective of the degree of prohormone processing. Trypsin treatment of plasma samples prior to RIA also served as a useful alternative to extraction by abolishing unspecific interference from plasma proteins in the radioimmunoassay.

The human proBNP sequence contains several sites for possible amino acid derivatizations and endoproteolytical cleavages (FIG. 1). Moreover, the prosequence contains a leucine zipper-like sequence motif that has been reported to induce oligomerizatlon of the N-terminal fragments of proBNP and proBNP In plasma (Seidler et al., 1999). This finding raises the possibility that oligomerized N-terminal proBNP fragments expose some regions while others are not accessible to antibody binding.

This may explain, at least in part, why the plasma concentration of N-terminal proBNP fragments in healthy and in heart failure patients published so far show considerable variation (Hunt et al., 1995 and 1997a,b; Schultz et al., 2001; Karl et al., 1999; Hughes et al., 1999; Campbell et al., 2000). Most methods are based on immunoassays using antisera raised against the N-terminus of proBNP, but precise definition of the epitope(s) has not always been reported. We have established the free N-terminus of human proBNP as the binding site by testing our antisera for binding to N-terminally truncated forms of human proBNP and to proBNP from other mammals with deviant N-terminal sequences. Furthermore, in a preferred embodiment of the present invention, we have found that trypsin treatment of plasma efficiently cleaves the endogenous N-terminal proBNP forms after the arginyl residue in position 21 (FIG. 4). We have thereby, in a preferred embodiment of the present invention, developed an assay that measures the N-terminal 1-21 fragment of proBNP from all forms of N-terminal proBNP with equilmolar potency. This assay corroborates that N-terminal proBNP fragments in healthy subjects circulate in the low picomolar concentration range in agreement with earlier reports (Hunt et al. 1995; Campbell et al., 2000).

The N-terminal proBNP concentrations in plasma were measured in groups with different age and gender. We did find a significant increase in plasma concentrations between age groups. For subjects under the age of 70 years, this was of no clinical importance for the calculation of an upper reference limit. We could not find any difference between genders. However, subjects >70 years of age without earlier cardiac disease or symptoms did show a substantial increase in the concentrations of N-terminal proBNP in plasma (FIG. 5), which suggests that they in fact had unidentified cardiac dysfunction or other medical conditions. When examining a second group of elderly >70 of age without objective signs of cardiac dysfunction, the N-terminal proBNP concentration was found to be lower when compared to the first group of elderly—but still not equal to the group of subjects less than 70 years of age. The N-terminal proBNP concentrations in the heart failure patients were all higher than the age-matched reference limit. Therefore, one of the many beneficial diagnostic uses of the assay of the present invention will be to exclude a diagnosis of cardiac impairment.

### Example 2

### Measurements of proBNP in Terminal Parenchymal Lung Disease Patients

Materials & Methods

Patients

Consecutive patients with a diagnosis of terminal parenchymal lung disease referred for lung transplantation evaluation between February 2000 and April 2001. Patients had to fulfil the following criteria: 1) Normal left ventricular function as assessed by two-dimensional echocardiography; 2) no significant coronary artery disease evaluated by angiography; 3) no renal impairment (serum creatinine a 130 gmol/L; and 4) no sustained arrhythmia, such as atrial fibrillation. Included were then 44 patients with terminal parenchymal lung disease with chronic obstructive pulmonary disease (COPD, n=19), emphysema with or without alfa-1 anti-trypsin deficiency (n=17) or pulmonary fibrosis (n=8). An additional 6 patients with primary pulmonary hypertension (PPH) were also evaluated. The study was approved by the local ethics committee (KF 01-307/99) and written—informed consent—for participation in the study was obtained from all patients.

Hemodynamics and Cardiac Function

Right and left heart catheterization was performed after an overnight fast through the femoral approach. Pressures were recorded from the right atrium (RAP mean), right ventricle (RVP systole and diastole), pulmonary artery (PAP systole, diastole and mean) and pulmonary artery wedge position (PCWP mean). Cardiac output (CO) was determined by either Fick's oxygen method using Deltatrac™ for oxygen uptake measurements or continuous thermo method using a Vigilance™ computer monitor. Pulmonary hypertension was defined as mPAP at rest greater than 20 mmHg (Rich et al., 1987). Cardiac index (CI) was calculated as CO/body surface area and pulmonary vascular resistance (PVR) as PAP mean—PCWP mean/CO. Left heart catheterization including left ventricular pressure measurements and ventriculography and coronary arteriography were performed after the right heart catheterization. Right ventricular ejection fraction was estimated by radionuclide imaging technique and left ventricular ejection fraction was evaluated using two-dimensional measurements in parasternal and apical 4-chamber. Segmental movement in the left ventricular wall was also described including paradoxical movement of the septum.

N-Terminal proANP and proBNP Concentrations in Plasma

During catheterization, 20 ml blood samples were carefully drawn through a catheter from the femoral vein and the trunk of the pulmonary artery. Blood was immediately transferred to a 10 ml tube containing potassium EDTA (1 mg/ml) and a 10 ml tube containing potassium EDTA and aprotinin (500 U/ml). Also, a 20 ml blood sample was obtained from the femoral artery. Samples were immediately centrifuged and plasma stored at −80° C. until analysis. For measurement of N-terminal proANP, we used a commercial kit (Biotop OY, Oulu, Finland). This radioimmunoassay (RIA) has a reported sensitivity of 30 pmoVL with expected concentrations of 110-600 pmol/L in healthy subjects (Kettunen et al., 1994). The plasma concentration of proBNP was determined as outlined above in Example 1.

Statistical Analysis

All data were expressed as median and range. Comparison of data from the different patient groups were performed using a non-parametric test (Krustal-Wallis) followed by Dunn's multiple comparison test. Log transformation was used to normallse the distributions of peptide concentrations before performing correlation (Pearson) or linear regression analysis. For comparison of plasma concentrations in the different vascular beds, repeated measures one-way analysis of variance was used followed by the Newman-Keulis multiple comparison test. P values less than 0.05 were considered significant.

Results

The characteristics of the 44 patients with terminal parenchymal lung disease divided into subgroups and the 6 patients with PPH are listed in Table 1.

TABLE 1
Patient characteristics

| | Emphysema | COPD | Fibrosis | PPH |
|---|---|---|---|---|
| No. patients (females/males | 17 (12/5) | 19 (11/8) | 8 (5/3) | 6 (5/1) |
| Age (years) | 54 (39-67) | 58 (42-69) | 56 (32-70) | 40 (24-54) |
| LEVF (%) | 60 (60-65) | 65 (55-70) | 65 (55-70) | 60 (60-65) |
| FVC (L) | 1.9 (1.1-4.1) | 1.9 (0.9-3.7) | 1.4 (1.2-2.2) | 3.4 (2.8-4.4) |
| FVC of expected (%) | 64 (40-103) | 57 (26-88) | 46 (27-72) | 98 (91-109) |
| FEV₁ (L/min) | 0.75 (0.47-2.00) | 0.78 (0.46-1.50) | 1.20 (0.48-1.40) | 2.80 (2.20-3.30) |
| FEV₁ of expected (%) | 27 (17-63) | 29 (19-73) | 35 (18-48) | 87 (80-103) |
| DL_{co} (mmol/min/kPa) | 2.8 (1.1-8.5) | 3.6 (0.5-5.0) | 2.1 (0.6-7.0) | 6.0 (4.7-7.6) |
| DL_{co} of expected (%) | 34 (11-90) | 40 (7-52) | 26 (17-65) | 66 (50-82) |

5 Values (median and range) for left ventricular ejection fraction (LVEF), forced vital capacity (FVC), forced expiratory volume in 1 s (FEV₁) and diffusing capacity (DL_{CO}) in the 50 subjects.
Hemodynamic Variables and Cardiac Function

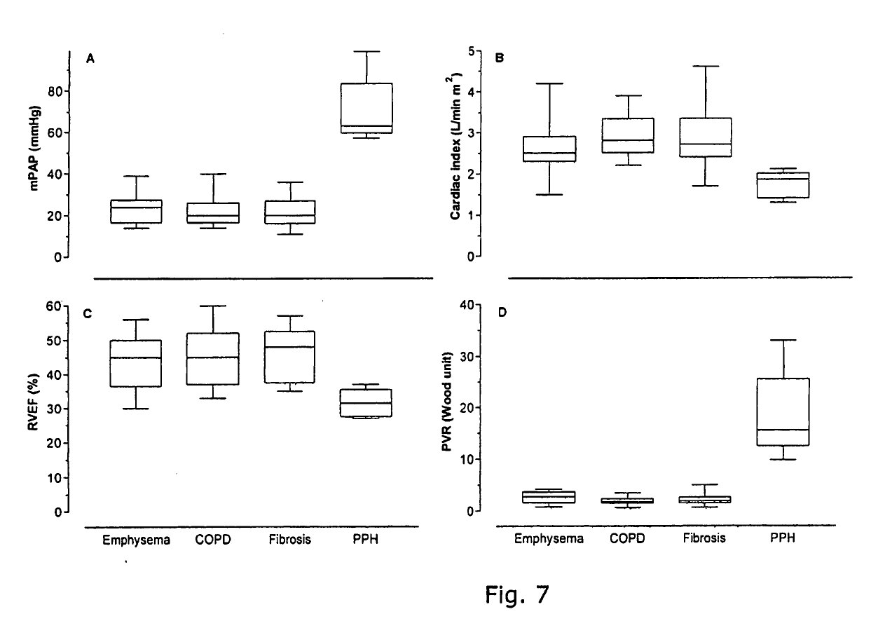

The mean pulmonary pressure, right ventricular ejection fraction, pulmonary vascular resistance and cardiac index did not differ between the 3 subgroups with parenchymal lung disease (FIG. 7). However, mean pulmonary pressure in these patients was elevated in 27 of 44 (67%) with a median mPAP of 20.5 (11-40) mmHg. Right ventricular ejection fraction was estimated to 48 (30-60)%, total pulmonary resistance 2.1 (0.7-5.0) Wood units and cardiac index calculated to 2.7 (1.5-4.6) L/min×m². In contrast, all patients with a diagnosis of primary pulmonary hypertension had highly elevated mean pulmonary pressures (63, 57-99) mmHg with low right ventricular ejection fractions (32, 27-37%). Pulmonary vascular resistance was increased (15.5, 9.8-33.0 Wood units) and cardiac index reduced (1.9, 1.3-2.1 L/min×M2) (FIG. 7). On echocardiography, paradox movement of the interventricular septum was recorded in all PPH patients but not in any of the patients with terminal parenchymal lung disease (data not shown).

Plasma N-Terminal proANP

The plasma concentrations of N-terminal proANP from the pulmonary artery were significantly higher than the upper reference limit in the parenchymal lung disease patients (664 (365-1620) vs 600 pmol/L, P<0.05) with no significant difference between the 3 subgroups (data not shown). Fourteen of 44 patients (32%) had elevated concentrations of N-terminal proANP despite a normal mean pulmonary pressure, whereas 11 of 44 (25%) had normal plasma concentrations of N-terminal proANP while also having elevated mPAP. No statistical relation of mean pulmonary pressure to the N-terminal proANP concentrations in plasma in any subgroups could be demonstrated. In the group of PPH patients, N-terminal proANP concentrations were 2-fold higher than in patients with terminal parenchymal lung disease (1186 (1008-1803) vs. 664 (365-1620) pmol/L, P<0.001).

Plasma proBNP

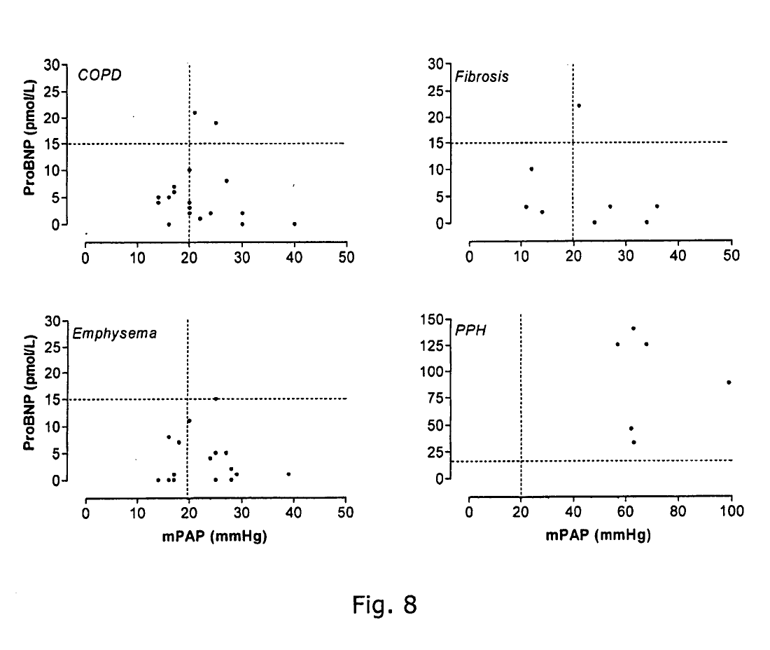

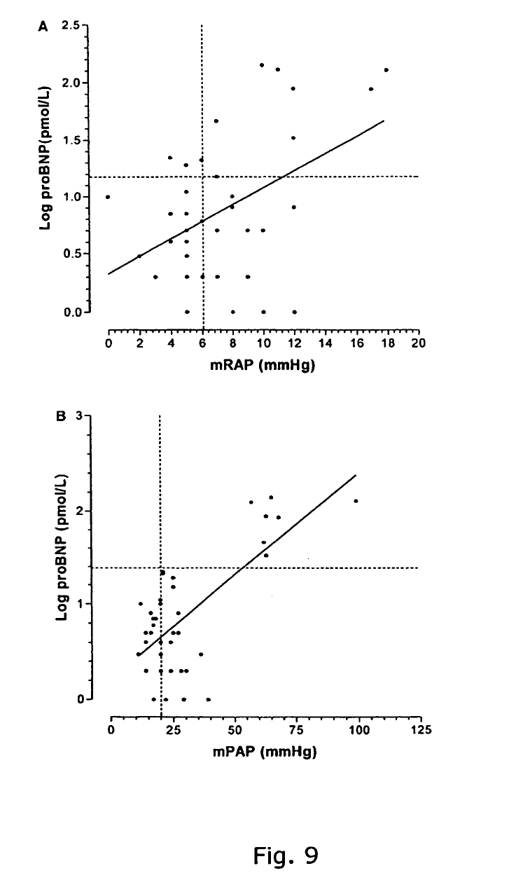

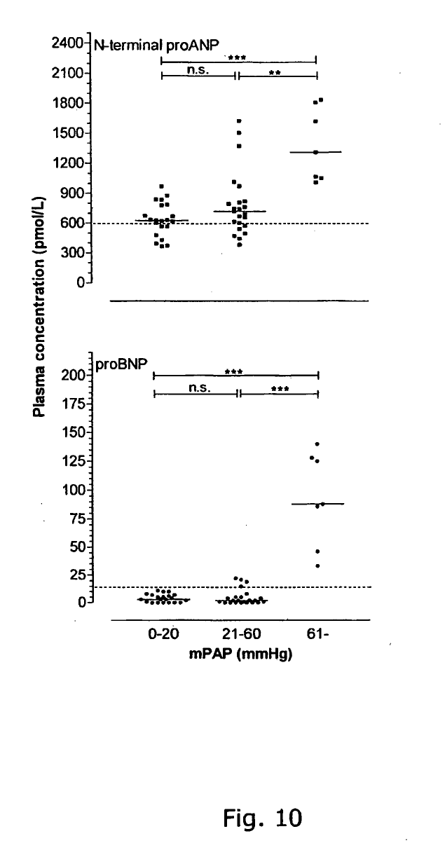

The results of plasma measurements from the pulmonary artery are shown in FIG. 8. In patients with terminal parenchymal lung disease and normal mPAP, the plasma proBNP concentration was always below the upper reference limit and only 4 of 27 patients (15%) with elevated mPAP had moderately increased concentrations of proBNP. Median plasma proBNP concentration in terminal parenchymal lung disease patients was 2.5 (0.0-22.0) pmol/L with no significant difference between the subgroups. In contrast, plasma proBNP In the PPH patients was increased more than 40-fold to 106.5 (22-140) pmol/L. No significant association between mPAP and plasma concentrations of proBNP was found in the parenchymal lung disease subgroups. Nevertheless, linear regression analysis disclosed a significant relationship of plasma proBNP concentrations to both mRAP and mPAP when including all parenchymal lung disease patients and the group with PPH (FIG. 9). N-terminal proANP and proBNP concentrations in all patients were likewise significantly correlated (r-0.47, P<0,0001). When patients were divided into 3 groups depending on mPAP, no difference in either N-terminal proANP or proBNP concentrations could be demonstrated with mPAP<60 mmHg (FIG. 10). Only the group of patients with mPAP>60 mmHg (consisting of 6 PPH patients and 1 with terminal parenchymal lung disease) had increased plasma propeptide concentrations.

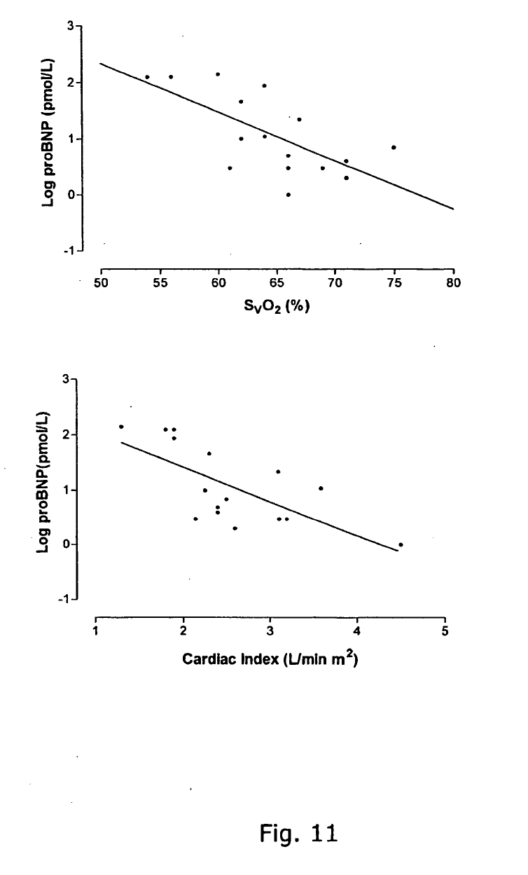

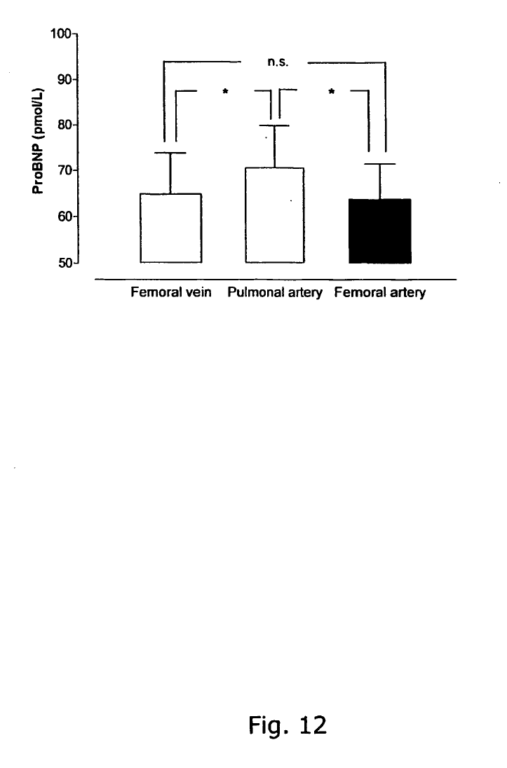

In 19 of 50 patients where oxygen saturation was determined in the pulmonary artery at catheterization, the concentration of proBNP in plasma proved dependent on pulmonal artery oxygen saturation as well as cardiac index in the patients (FIG. 11). Finally, the concentrations of proBNP in 3 different vessels were compared. In patients with elevated proBNP concentrations (4 patients with terminal parenchymal lung disease and 6 patients with PPH, n=10), a significantly higher proBNP concentration could be demonstrated in the pulmonary artery as compared to both the concentrations in the femoral vein and the femoral artery (FIG. 12, P<0.05).

Discussion

Patients with terminal parenchymal lung disease and normal left ventricular function have no increase in plasma proBNP and there is no relation between mean pulmonary artery pressure and plasma peptide concentrations in these patients. In contrast, the group of patients with PPH displayed elevated plasma concentrations of N-terminal proBNP (2-fold) and plasma proBNP increased more than 40-fold (FIG. 12). A relation of mean pulmonary artery pressure and proBNP concentrations could only be demonstrated when also this group of patients was included.

The higher proBNP concentration in plasma sampled from the pulmonary artery confirms that the peptide measurements represent cardiac secretion (FIG. 12). However, the results do not disclose the exact origin of synthesis and secretion within the heart. As elevated concentrations of proBNP was almost exclusively detected in the group of patients with PPH, it is striking that only these patients also displayed a paradox movement of the interventricular septum. In an experimental model of acute right ventricular overload with the central pulmonary artery pressure elevated 2-fold, tissue samples from the interventricular septum did not show ANP and BNP mRNA to be upregulated after distal pulmonary arterial banding (Adachi et al., 1995). All patients with primary pulmonary hypertension in the present study had chronically elevated pulmonary artery pressure more than 3-fold. It therefore seems conceivable that involvement of the interventricular septum in severe right ventricular overload may contribute to the elevated plasma concentrations of natriuretic peptides. The significant concentration gradient between the pulmonary artery and femoral artery furthermore indicates that proBNP may be metabolised in the lung. The lung has been suggested to be a major clearance organ for the C-terminal, bloactive BNP peptide by expression studies of the metabolising enzyme neutral endoepeptidase (Li et al., 1995).

The major symptoms of terminal parenchymal terminal lung disease are severe dyspnea with a concomitant hypoxia. Clinical worsening of such chronic respiratory condition often leads to admissions to emergency departments, where the main presenting symptom is dyspnea, which is also a cardinal symptom of congestive heart failure. Studies of natriuretic peptide measurements as discriminative biochemical markers of cardiovascular or pulmonary causes of dyspnea are now accumulating and the findings are highly encouraging (Morrison et al., 2002; Harrison et al., 2002). Our data demonstrates that even in terminal patients with isolated parenchymal disease eligible for lung transplantation, plasma concentrations of proBNP are normal even in the presence of a moderate degree of pulmonary hypertension.

The present study reveals that plasma concentrations of proBNP in patients with terminal parenchymal pulmonary disease referred for lung transplantation is not elevated even in the presence of pulmonary hypertension. Secondly, proBNP appears to be secreted mainly from the heart in patients with vascular pulmonary disease. The findings thus exclude natriuretic-peptides as markers of pulmonary hypertension in parenchymal lung disease patients but corroborate the idea that natriuretic peptides can be used as discriminative markers between pulmonary or cardiovascular causes of dyspnea. Accordingly, the methods of the present invention can be used as a simple diagnostic test to distinguish between pulmonary and cardiovascular causes of dyspnea.

### Example 3

### Cardiac proBNP Expression is Increased in Myocardial Ischemia

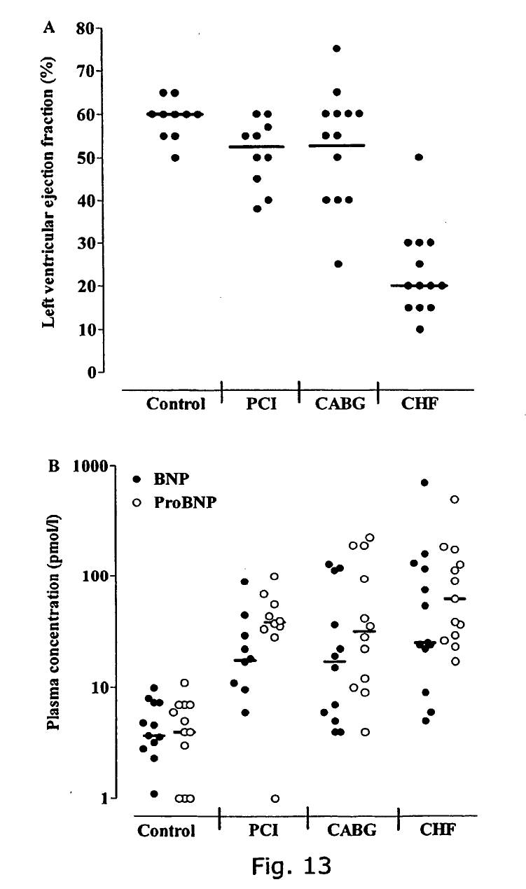

The cardiac BNP secretion was examined in plasma from coronary artery disease patients undergoing coronary bypass grafting surgery or percutaneous transluminal intervention therapy. Ventricular function was assessed by ventriculography and was not significantly decreased in the ischemic patients when compared to a group of normal individuals (FIG. 13A). However, the plasma BNP and proBNP concentrations in the coronary artery bypass grafting surgery patients were markedly increased (BNP 5-fold, P<0.01, proBNP 8-fold, P<0.01) when compared with a control group without coronary artery disease (FIG. 13B). Similarly to the coronary artery bypass grafting surgery patients, another group of patients undergoing percutaneous intervention therapy had normal left ventricular ejection fraction (FIG. 13A) and markedly increased plasma BNP and proBNP concentrations (BNP 5-fold, P<0.01, proBNP 10-fold, P<0.01) compared with the control group (FIG. 13B). Of note, the plasma BNP and proBNP concentrations in the coronary artery disease patients did not significantly differ from those found in a group of congestive heart failure patients with severely reduced left ventricular ejection fraction (FIG. 13B).

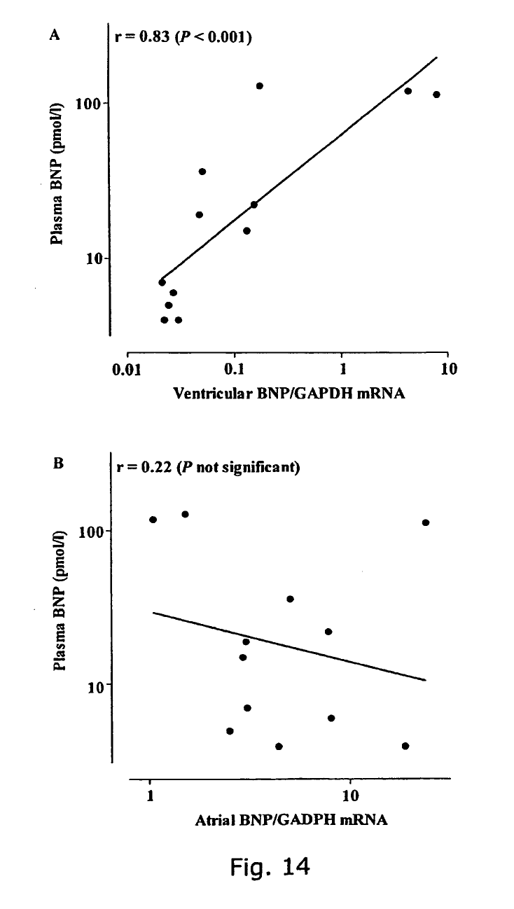

Quantitative analysis of cardiac BNP mRNA in atrial and ventricular biopsies from coronary artery disease patients undergoing coronary artery bypass grafting surgery revealed a positive association between ventricular BNP mRNA expression and plasma BNP concentration (r=0.83, P<0.001, FIG. 14A) and plasma proBNP concentration (r=0.78, P<0.001). In contrast, plasma BNP and proBNP concentrations were not associated with the atrial BNP mRNA expression (FIG. 14B) despite that the atrial BNP mRNA expression was almost 6-fold higher in atrial than ventricular biopsies.

1. A method for determining the concentration of a BNP precursor, or fragments thereof, in a sample obtained from a mammal, the method comprising treating the sample with an agent that cleaves the BNP precursor, and exposing the sample to an antibody that specifically binds to the cleaved product.

2. A method according to claim 1, wherein the cleaved product is any fragment of proBNP which can bind an antibody.

3. A method according to claim 1, wherein the fragment is at least 6 amino acids in length.

4. A method according to claim 1, wherein the antibody binds the N-terminus or the C-terminus of the cleaved product.

5. A method according to claim 4, wherein the antibody binds the N-terminus of proBNP1-21.

6. A method according to claim 1, wherein the sample is selected from the group consisting of blood, serum, plasma, urine and a biopsy of the heart or other organs.

7. A method according to claim 1, wherein the agent is any molecule that cleaves the BNP precursor to produce a fragment which can bind an antibody.

8. A method according to claim 7, wherein the agent is an enzyme.

9. A method according to claim 8, wherein the enzyme is an serine protease selected from the group consisting of trypsin, furin, corin, yeast Kex2, prohormone convertase-1 and prohormone convertase-2.

10. A method according to claim 1 wherein the agent that cleaves the precursor cleavers at basic amino acids and the antibody specifically binds an N-terminus of proBNP1-21.

11. A method of predicting or diagnosing a cardiac disease, the method comprising performing the method of claim 1, wherein elevated levels of antibody binding are indicative of cardiac dysfunction.

12. A method according to claim 11, wherein the cardiac dysfunction is selected from the group consisting of congestive heart failure, impaired function of the left ventricle, cardiac failure after myocardial infarction, arrhytmogenic right ventricular dysplasia, chronic respiratory disease due to tuberculosis, congenital heart disease, obstructive hypertrophic cardiomyopathy, predicting mortality in elderly and cardiac related acute dyspnea.

13. A method of predicting or diagnosing a cardiac transplant rejection episode, the method comprising performing the method according to claim 1, wherein an elevated level of antibody binding are indicative of a rejection episode.

14. A method to distinguish between pulmonary and cardiovascular causes of dyspnea, the method comprising performing the method according to claim 1, wherein elevated levels of antibody binding are indicative of cardiovascular causes of dyspnea.

15. A method of predicting or diagnosing an ischemic heart disease, the method comprising performing the method according to claim 1, wherein elevated levels of antibody binding are indicative of a ischemic heart disease.

16. A method according to claim 11, wherein an elevated level of antibody binding level is a levels above 15 pmol/L.

17. A method for evaluating the effect of coronary angiography, the method comprising performing the method according to claim 1, wherein the time of (blood) sampling is correlated in relation to invasive assessment.

18. A method according to claim 12, wherein an elevated level of antibody binding levels is a level above 15 pmol/L.

19. A method according to claim 13, wherein an elevated level of antibody binding levels is a level above 15 pmol/L.

20. A method according to claim 14, wherein an elevated level of antibody binding levels is a level above 15 pmol/L.

21. A method according to claim 15, wherein an elevated level of antibody binding levels is a level above 15 pmol/L.


SEQUENCE LISTING (7 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 131 aa, Sus scrofa
MGPRMALPRVLLLLFLHLLLLGCRSHPLGGAGLASELPGIQELLDRLRDRVSELQAERTDLEPLRQDRGLTEAWEAREAAPTGVLGPRSSIFQVLRGIRSPKTMRDSGCFGRRLDRIGSLSGLGCNVLRRY

SEQ ID NO: 3 - protein, 121 aa, Mus musculus
MDLLKVLSQMILFLLFLYLSPLGGHSHPLESPSQSPEQFLMQKLLELIREKSEEMAQRQLLKDQGLTKEHLKRVLRSQGSTLRVQQRPQNSKVTHISSCFGHKIDRIGSVSRLGCNALKLL

SEQ ID NO: 4 - protein, 32 aa, Homo sapiens
SPKMVQGSGCFGRKMDRISSSSGLGCKVLRRH

SEQ ID NO: 5 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 6 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 7 - protein, 21 aa, Homo sapiens
HPLGSPGSASDLETSGLQEQR